(19) United States
(12) Patent Application Publication
(10) Pub. No.: US 2025/0097605 A1
(43) Pub. Date: Mar. 20, 2025

(54) DIGITAL AUTOCORRELATOR

(71) Applicant: MIAMI UNIVERSITY, OXFORD, OH (US)

(72) Inventor: KARTHIK VISHWANATH, OXFORD, OH (US)

(73) Assignee: MIAMI UNIVERSITY, OXFORD, OH (US)

(21) Appl. No.: 18/888,800

(22) Filed: Sep. 18, 2024

Related U.S. Application Data
(60) Provisional application No. 63/583,485, filed on Sep. 18, 2023.

(51) Int. Cl. H04N 25/773

(52) U.S. Cl. CPC H04N 25/773

(57) ABSTRACT

Various technologies described herein pertain to systems and associated methods that autocorrelate temporal signals and output an autocorrelated signal upon which vascular perfusion in tissue may be determined. The system includes a sampler that receives a first indication and a second indication at a lag time after the first indication from an indication generator. The indication generator can be a photodetector, such as in diffuse correlation spectroscopy applications of the system. The sampler generates and transmits first and second digitized indications to a processing unit of the system. The processing unit employs an algorithm that includes generating an autocorrelated indication based on the first digitized indication, the second digitized indication, and the lag time. The processing unit outputs the autocorrelated indication such that the autocorrelated indication may be used to non-invasively measure vascular perfusion in tissue.

### TECHNICAL FIELD

The present invention is in the field of systems and methods of digital signal processing. More particularly, the present invention provides systems and methods for processing temporal signals received from photodetectors, such as in a biomedical optics application of the disclosed systems and methods.

### SUMMARY

The following is a brief summary of subject matter that is described in greater detail herein. This summary is not intended to be limiting as to the scope of the claims.

Disclosed herein are various technologies pertaining to systems and methods for autocorrelating temporal signals. Autocorrelation of temporal signals is a signal processing technique useful in many scientific areas. Temporal autocorrelations of signals require that input signals be sampled with high-fidelity so that there is little to substantially no loss in post-processed signal quality or information. In biomedical optics, diffuse correlation spectroscopy (DCS) uses signal autocorrelation to non-invasively measure vascular perfusion in tissue at depths of a few millimeters to a few centimeters or more under a surface, such as a skin surface. For DCS applications, inputs to each channel of an autocorrelator is obtained from photodetectors, such as single photon avalanche diodes (SPADs). The SPAD is a single photon detector and operates in Geiger (or photon counting) mode. Thus, TTL (transistor-transistor logic) pulses are outputted—where each pulse (in a fixed window of time, e.g., ˜5-10 ns) represents whether or not a photon detection event occurs. Outputs from the detected photocurrents emerge as a digitized transistor-to-transistor logic (TTL) stream, with countrates typically in the range of about 20 MHz to about 40 MHz. In certain applications, sampling frequency of the signal from the SPAD is adjusted/matched to be commensurate with the SPAD detector's timing. Specifically, DCS requires calculation of measured, temporal, photon-intensity correlations. In other words, DCS requires correlation of a first photon intensity signal measured at a first time and a second photon intensity signal measured at a second time later than the first time, where a difference between the second time and the first time defines a lag time. Autocorrelating the first and second signals can enable the detection of a repeating pattern common to the first and second signals that would otherwise be obscured by noise in the first and second signals. In DCS applications, the repeating pattern can be indicative of a characteristic of blood flow through tissue, such as volumetric flow rate.

According to an exemplary embodiment, a system for autocorrelating temporal signals comprises a sampler and a processing unit. The sampler is configured to receive a first signal from a signal generator (e.g., from a photodetector that generates a signal based on an intensity of a photon) and to receive a second signal from the signal generator at a lag time after the first signal is received by the sampler. For example, the first signal and the second signal can be continuous signals and the sampler can be configured to sample at least two signals from the continuous signals. The sampler is further configured to generate a first digitized signal based on the first signal and to generate a second digitized signal based on the second signal. The processing unit comprises a processor and a memory, wherein the memory has machine-readable instructions stored thereon, wherein when executed by the processor, cause the processor to perform certain acts. The acts comprise receiving the first and second digitized signals. The acts further comprise generating an autocorrelated signal based on the first digitized signal, the second digitized signal, and the lag time. The acts additionally comprise outputting the autocorrelated signal. The autocorrelated signal may be used to non-invasively measure one re more medically relevant variables, such as vascular perfusion in tissue. For example, a frequency and/or an amplitude of the autocorrelated signal may be indicative of a characteristics of blood flow through the tissue.

According to an exemplary embodiment, a system for generating autocorrelation indications is provided. The system comprises a detector, a sampler, and a processing unit. The detector is configured to detect a first presence or absence of a first photon, generate a first presence indication based on the first presence or absence of the first photon, transmit the first presence indication, detect a second presence or absence of a second photon, generate a second presence indication based on the second presence or absence of the second photon, and transmit the second presence indication. The sampler is configured to receive the first presence indication from the detector, receive the second presence indication from the detector, generate a first digitized presence indication based on the first presence indication, and generate a second digitized presence indication based on the second presence indication. The processing unit comprises a processor, a receiver, and a non-transitory computer-readable medium storing machine executable instructions, wherein when executed by the processor, cause the processor to perform acts comprising receiving the first digitized presence indication and the second digitized presence indication, and generating an autocorrelation indication based on the first digitized presence indication and the second digitized presence indication.

According to an exemplary embodiment, a method for generating autocorrelation indications is provided. The method comprises detecting a first presence or absence of a first photon by a detector; generating a first presence indication based on the first presence or absence of the first photon by the detector; transmitting the first presence indication by the detector; detecting a second presence or absence of a second photon by the detector; generating a second presence indication based on the first presence or absence of the second photon by the detector; transmitting the second presence indication by the detector; receiving the first presence indication by a sampler; receiving the second presence indication by the sampler; generating a first digitized presence indication based on the first presence indication by the sampler; generating a second digitized presence indication based on the second presence indication by the sampler; receiving the first digitized presence indication and the second digitized presence indication by a processing unit; generating an autocorrelation indication based on the first digitized presence indication and the second digitized presence indication; and outputting the autocorrelation indication.

Exemplary embodiments of the system facilitate development and scaling in biophotonics applications using DCS, such as monitoring human brain function in real-time, for example, by monitoring blood flow in regions of the brain in real-time. Systems performing DCS typically require the use of an autocorrelator per channel of the DCS system. Costs for conventional autocorrelation systems scale non-linearly with the number of channels required for the DCS application. Additionally, it becomes difficult to scale-up the number of channels once a particular DCS system is built with an arbitrary number of channels in a conventional autocorrelation system. Exemplary embodiments of the system described herein overcome or mitigate the aforementioned problems, for example, by way of being a programmable, multi-channel autocorrelation system.

The above summary presents a simplified introduction in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

These and other features of the present disclosure will become better understood with regard to the following description and accompanying drawings in which:

FIG. 1 illustrates an exemplary system for autocorrelating temporal signals.

FIG. 2 is a block diagram representing an exemplary algorithm.

FIG. 3 is a graph of test results of a sampler of an exemplary system for autocorrelating temporal signals.

FIG. 4 is a graph of test results of a sampler of an exemplary system for autocorrelating temporal signals.

FIG. 5 is a graph of test results of a sampler of an exemplary system for autocorrelating temporal signals.

FIG. 6 illustrates an exemplary computing system for use with the general inventive concepts.

### DETAILED DESCRIPTION

Various technologies pertaining to systems and methods for autocorrelating temporal signals are described in detail herein. In the following description, for purposes of explanation, numerous specific details are set forth in order to provide a thorough understanding of one or more aspects. It may be evident, however, that such aspect(s) may be practiced without these specific details. In other instances, well-known structures and devices are shown in block diagram form in order to facilitate describing one or more aspects. Further, it is to be understood that functionality that is described as being carried out by certain system components may be performed by multiple components. Similarly, for instance, a component may be configured to perform functionality that is described as being carried out by multiple components.

The term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary” is intended to mean serving as an illustration or example of something and is not intended to indicate a preference.

The acts described herein may be computer-executable instructions that can be implemented by one or more processors and/or stored on a computer-readable medium or media. The computer-executable instructions can include a routine, a sub-routine, programs, a thread of execution, and/or the like. Still further, results of acts of the methodologies can be stored in a computer-readable medium, displayed on a display device, and/or the like. Additionally, the computer-readable instructions may be written in different computer programing languages including but not limited to: assembly language, C, C++, Java, and/or Python.

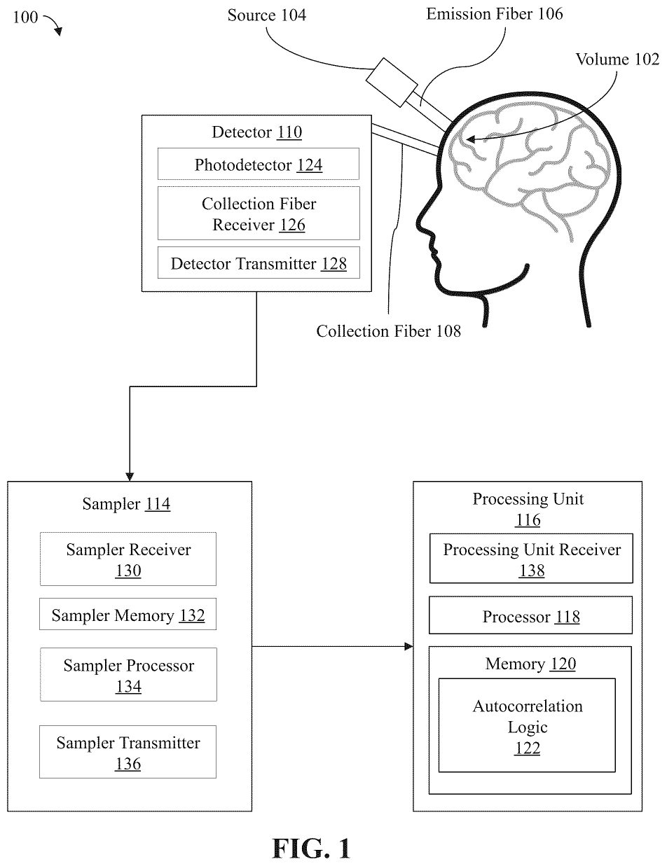

With reference to FIG. 1, an exemplary system 100 for autocorrelating temporal signals is shown. The system 100 comprises a detector 110, a sampler 114, and a processing unit 116. The detector 110 comprises a photodetector 124, a collection fiber receiver 126, and a detector transmitter 128. The photodetector 124, the collection fiber receiver 126, and the detector transmitter 128 are in electrical connection. The collection fiber receiver 126 is configured to connect to a collection fiber 108. The sampler 114 comprises a sampler receiver 130, a sampler memory 132, a sampler processor 134, and a sampler transmitter 136. The sampler receiver 130, the sampler memory 132, the sampler processor 134, and the sampler transmitter 136 are in electrical connection. The processing unit 116 comprises a processing unit receiver 138, a processor 118, and a memory 120. The processing unit receiver 138, the processor 118, and the memory 120 are in electrical connection. The detector 110 can be in wired or wireless communication with the sampler 114. The sampler 114 is configured to receive a first signal (or a first presence indication) from the detector 110 indicating if a photon is present or absent by receiving the signal from the detector transmitter 128 through the sampler receiver 130 by a physical electrical connection or a wireless connection. The sampler 114 is further configured to receive a second signal (or a second presence indication) from the detector 110 indicating if a photon is present or absent at a default lag time before or after the first signal is received by the sampler 114. Furthermore, the sampler 114 is configured to generate a first digitized signal (or a first digitized presence indication) based on the first signal. The sampler 114 is further configured to generate a second digitized signal (or a second digitized presence indication) based on the second signal. The sampler 114 is further configured to be in wired and/or wireless electrical communication with the processing unit 116 by sending messages from the sampler transmitter 136 to the processing unit receiver 138 through a physical electrical connection or a wireless connection. In one embodiment of the invention, the first signal and/or the second signal correspond to the presence or absence of photons detected by the photodetector 124. For example, the detector 110 can generate and transmit a message indicating the presence of one photon detected by the photodetector 124 and/or a different message indicating the absence of any photons detected by the photodetector 124. Similarly, in other embodiments of the invention, the detector 110 can be configured to generate and transmit a message indicating the presence of multiple photon (and/or the photon count) by the photodetector 124.

In certain embodiments, the system 100 is configured to analyze a volume 102 (e.g., a volume of tissue in which vascular perfusion is to be measured via DCS). In certain embodiments, the system 100 further comprises a photon source 104 in optical communication with an emission fiber 106 (e.g., a single mode fiber), and a detector 110 (e.g., a photodetector 110) in optical communication with the collection fiber 108. The photon source 104 generates photons, and the emission fiber 106 guides these photons to impinge upon the volume 102. The volume 102 reflects (i.e., scatters) at least some of the photons. At least some of the reflected photons are collected by the collection fiber 108 (e.g., a single mode fiber). The collected photons are detected at varying times by the detector 110. In such embodiments, the detector 110 is capable of detecting if a photon is collected by the collection fiber 108 and capable of generating different signals based on if a photon is detected or not. In an alternative embodiment, the detector 110 is capable of detecting the intensity and/or the number of photons collected by the collection fiber 108 and capable of generating different signals based on the level and/or number of photons detected. According to exemplary embodiments, the detector 110 generates a first voltage according to the Transistor-Transistor-Logic (TTL) protocol based on a first detected photon presence or absence and a second voltage according to the TTL protocol based on a second detected photon presence or absence, where the second detected photon presence is detected by the detector 110 at a default time lag after the first detection is completed. In one embodiment, the detector 110 is configured to generate a voltage at around 0.0V to indicate there is no photon detected and a voltage at around 3.3V to 5V to indicate a photon is detected. The first and second voltages are transmitted to the sampler 114. The sampler 114 receives the first voltage and the second voltage and converts the first and second voltages into a first digitized binary presence indication and a second digitized binary presence indication respectively. In one embodiment, the sampler 114 is configured to receive and convert (or sample) the voltage at 10 MHz. In one embodiment, the digitized binary presence indications are configured to be 00 for no photon detected and 01 for one photon detected. In such embodiment, the binary presence indications are stored in a file with a binary data format. The first and second digitized binary presence indication are transmitted to the processing unit 116. The processing unit 116 generates an autocorrelated signal (or an autocorrelation indication) based on the first digitized binary presence indication and the second digitized binary presence indication by assuming the first digitized binary presence indication is at time 0 and the second digitized binary presence indication is at time 1. In alternative embodiments, the lag time is a default lag time that is predetermined and configured to be the same for the detector 110, sampler 114, and the processing unit 116. In such embodiments, the default time lag is stored in the memory 120. Further, in such embodiments, the detector 110 is configured to label each presence indication based on the increment of the default time lag, or, the sampler 114 is configured to label each presence indication or digitized presence indication based on the increment of the default time lag, and/or the processing unit 116 is configured to label each digitized presence indication based on the increment of the default time lag. In some embodiments, the time lag can be manually adjusted after and/or synchronized with the detector 110, sampler 114, and/or the processing unit 116. The processing unit 116 outputs the autocorrelated signal. In alternative embodiments, the detector 110 can be configured to generate other signals instead of the first and second voltage to indicate the presence of a photon. Similarly, the sampler 114 is configured to generate other signals instead of the first and second digitized binary presence indication to indicate the different signals generated by the detector 110.

In an alternative embodiment, the detector 110 is configured to detect the number of photons collected by the collection fiber 108 and transmit a first intensity signal and/or a first indication of the number of photons detected based on a first detection and a second intensity signal and/or a second indication of the number of photons detected based on a second intensity signal. The sampler is configured to generate a first digitized intensity signal based on the first intensity signal and a second digitized intensity signal based on the second intensity signal. The processing unit 116 is configured to generate an autocorrelated signal based on the digitized intensity signals and/or store the autocorrelation indication.

In alternative embodiments, the sampler 114 is configured to sample a period of time. In such embodiments, the sampler 114 continuously receives the signals sent by the detector 110 and continuously generate digitized representations of the received signals. The generated signals are stored in the sampler memory 132 and transmitted to the processing unit 116. The processing unit 116 is configured to continuously generate autocorrelated signal based on the digitized representations at each default time intervals or at a predetermined time intervals. The processing unit 116 is optionally configured to generate a graphical representation of the autocorrelated signals.

In alternative embodiments, the sampler is configured to modify the presence indication and/or intensity indication before or after conversion.

In exemplary embodiments, the detector 110 is a single photon detector (i.e., a photodetector capable of detecting a single photon) such as a single photon avalanche diode (SPAD). Further, the detector 110 is configured to generate and/or output an electrical representation of an intensity of a photon detected by the detector 110. Furthermore, the detector 110 may output the electrical representation in at extremely short time intervals so that it could be regarded as or processed as a continuous signal, such as an analog signal. Accordingly, the detector 110 is an exemplary embodiment of a signal generator as described herein.

For certain applications of the system 100, such as for DCS applications, a temporal dynamic range of the sampler 114 may be required to span several orders of magnitude. For example, the sampler 114 can generate the first and/or second digitized signals by sampling the first signal and/or second signal at a frequency of at least about 10 MHz or higher. In alternative embodiments, the sampler 114 can sample the first and/or second signals at a frequency or a sub-range of frequencies in the range of about 10 MHz to about 80 MHz or higher. For example, the sampler 114 can sample at a frequency or a sub-range of frequencies in the range of about 10 MHZ to about 30 MHz, about 10 MHz to about 20 MHz, about 20 MHz to about 40 MHz, etc. Moreover, the sampler 114 can generate the first and/or second digitized signals by sampling the first and/or second signals for a sampling period of at least about 30 seconds or longer. In embodiments, the sampler 114 can sample for a sampling period or a sub-range of sampling periods in the range of about 30 seconds to about 600 seconds or higher. For example, the sampler 114 can sample for a sampling period or a sub-range of sampling periods in the range of about 60 seconds to about 600 seconds. In such embodiments, the sampling time and lag time is configured to be adjustable by the user. When sampling, the sampler 114 is optionally configured to buffer the signals received in sampler memory 132. The sampler processor 134 is configured to convert the TTL or other signals to binary signals (or other messages indicating the presence or number of photons) that can be stored in sampler 132 or other data storage devices. In alternative embodiments, the sampler 114 is configured to transmit the binary signals (or other messages indicating the presence or the number of photons) as they are generated or wait until all the signals are processed and transmit the aggregate of the signals.

The system 100 is configured to transmit a digitized signal from the sampler 114 to the processing unit 116. In other words, the processing unit 116 is configured to receive a digitized signal (e.g., a first digitized signal and/or a second digitized signal) from the sampler 114. The processing unit 116 may also receive a lag time from the sampler 114 or retrieve a default lag time that is preconfigured into both the detector 110 and/or the sampler 114. In embodiments, the sampler 114 and the processing unit 116 are in wired communication, for example, by way of a USB connection. Accordingly, the sampler 114 can transmit the digitized signal to the processing unit by way of the USB connection. In other embodiments, the sampler 114 and the processing unit 116 are in wireless communication, for example, by way of a Bluetooth-based connection. Accordingly, the sampler 114 can wirelessly transmit the digitized signal to the processing unit by way of the Bluetooth-based connection. Moreover, the sampler 114 can be configured to compress the digitized signal prior to transmitting the digitized signal to the processing unit 116. Accordingly, the processing unit 116 may be configured to decompress a compressed signal prior to further processing of the signal. In some embodiments, the processing unit 116 is configured to generate autocorrelation indication based on any two adjacent digitized presence indication. In other embodiments, the processing unit 116 is configured to generate autocorrelation indication based on digitized presence indication selected by the user. In other embodiments, the processing unit 116 is configured to generate autocorrelation indication based on each digitized presence indication at first sampling period compared to each digitized presence indication at the second sampling period.

The processing unit 116 is configured to autocorrelate the digitized signal to form an autocorrelated signal. For example, the processing unit 116 is configured to generate an autocorrelated signal based on a first digitized signal, a second digitized signal, and/or a lag time. Further, the processing unit 116 can be configured to output the autocorrelated signal, for example, for use in non-invasively measuring vascular perfusion in tissue. For example, a frequency and/or an amplitude of the autocorrelated signal may be indicative of a parameter of blood flow through the tissue. In other embodiments, the sampler 114 does not transmits a lag time but instead transmits time stamps of each digitized signal. In such embodiments, the processing unit 116 receives the time stamps of each digitized signal and calculates the lag time based on the differences.

As shown in FIG. 1, the processing unit 116 comprises a processor 118 and a memory 120. The memory 120 has executable instructions stored thereon. Certain executable instructions may be executed according to autocorrelation logic 122. In certain embodiments, the executable instructions and/or the autocorrelation logic 122 comprise C/C++ code. It is appreciated that the autocorrelation logic 122 may be loaded onto the memory 120 by way of a wireless connection, for example a Bluetooth-based connection or the like; a wired connection, for example a USB connection or the like; or an external storage medium, for example, a USB flash drive, an SD card, or the like. It is also appreciated that the autocorrelation logic 112 may comprise other computer programing language including but not limited to Assembly Language, Java, and/or Python. When the executable instructions are executed by the processor 118, the processor 118 is caused to perform certain acts. The acts can comprise receiving first and second digitized signals. The acts can further comprise calculating, measuring, or otherwise determining a time lag (e.g., a time lag between first and second signals upon which the first and second digitized signals are based). The acts can further comprise transmitting the time lag (e.g., by a photodetector and/or a sampler) and/or receiving the time lag (e.g., by a processor). The acts can further comprise generating an autocorrelated signal based on the first digitized signal, the second digitized signal, and/or the time lag. The acts can additionally comprise outputting the autocorrelated signal. The autocorrelated signal may be used by the processing unit 116 or another processor to measure and/or output a measurement of one or more medically relevant variables, such as a vascular perfusion in a tissue. For example, a frequency and/or an amplitude of the autocorrelated signal may be indicative of a parameter of blood flow through the tissue. In embodiments, the processing unit 116 comprises a minicomputer (e.g., a Raspberry Pi or the like), a laptop computer, a desktop computer, a cloud-based processor, or a combination thereof.

The system 100 can include a source 104, an emission fiber 106, a collection fiber 108, and a detector 110, as described herein. In embodiments of the system 100, more than one collection fibers 108, and/or more than one detectors 110 may be included in the system 100. The plurality of sources 104 can generate a plurality of input photon source signals. Each of the collection fibers 108 can collect a reflected photon signal. Accordingly, each of the detectors 110 can detect the collected photon signal. When an input photon source signal is coupled to a detected photon signal, a channel of the system 100 is formed. Moreover, a channel of the system 100 may comprise a pair of first and second digitized signals, as described herein. In an embodiment of the system 100 having a plurality of channels where each channel provides a stream of digitized SPAD outputs, a processing unit 116 is configured to generate an autocorrelated signal based on the plurality of channels (i.e., based on the plurality of pairs of first and second digitized signals) and a sampler 114 that is configured to convert the signals into digitized representation of the signals. In some embodiments, the digitized representation is stored in according to a predetermined protocol to indicate which channel(s) the signal(s) came from. Accordingly, each pair of first and second digitized signals can correspond to a detector 110 of the plurality of detectors 110. In embodiments, the plurality of detectors 110 is a SPAD array containing more than one SPAD, for example, on a silicon chip. In embodiments, the sampler 114 can generate a digitized signal(s) by sampling a signal corresponding to each channel a frequency of at least about 10 MHz or higher per channel, such as at least about 20 MHz or higher per channel.

In embodiments of the system 100, one or more collection fibers 108 can be coupled to one or more detectors 110. Each channel of the system 100 corresponds to a pair comprising an input and an output. For example, each source 104 generates input optical signals that are transmitted to the volume 102 by way of emission fiber 106. The volume 102 (e.g., moving blood cells within the volume 102) scatters and/or reflects at least some of the input optical signals to form output optical signals that can be collected by one or more collection fibers 108 and transmitted to one or more detectors 110. Such a pairing of an input signal and an output signal comprises a channel of the system 100. In embodiments, the system 100 can comprise multiple channels and the sampler 114 and/or the processing unit 116 can be configured to parallelly sample and/or parallelly process (i.e., autocorrelate) data corresponding to multiple channels.

In an embodiment of the system 100 having two (2) channels, include a first detector 110a coupled to a first collection fiber 108a and a second detector 110b coupled to a second collection fiber 108b. The pairing of a first input from the first source 104a with a first output detected by the first detector 110a comprises a first channel of the system 100. The pairing of a second input from the second source 104b with a second output detected by the second detector 110b comprises a second channel of the system 100. In other words, a plurality of channels in the system 100 may be formed based upon a quantity of pairs comprising a source 104 and a detector 110 and/or based upon a quantity of pairs comprising an emission fiber 106 and a collection fiber 108 in the system 100. For example, an eight (8) channel system 100 may comprise eight (8) detectors 110.

In other embodiments, a two (2) channel system 100 may comprise a single source 104 coupled to an emission fiber 106 and further comprise a first detector 110a coupled to a first collection fiber 108a and a second detector 110b coupled to a second collection fiber 108b. The volume 102 (e.g., moving blood cells within the volume 102) scatters and/or reflects at least some of the input optical signal from the source 104 to form output optical signals that can be collected by the first and second collection fibers 108a, 108b and detected by the first and second detectors 110a, 110b. In such embodiments, the pairing of a first input from the source 104 with a first output detected by the first detector 110a comprises a first channel of the system 100, and the pairing of the first input from the source 104 with a second output detected by the second detector 110b comprises a second channel of the system 100. In other words, a plurality of channels in the system 100 may be formed based upon a quantity of detectors 110 and/or collection fibers 108 in the system 100. For example, an eight (8) channel system 100 may comprise eight detectors 110 and any suitable number of sources 104, such as three (3) sources 104.

Through the above operation of the exemplary system 100, certain advantages are realized. For example, since the processing unit 116 performs signal autocorrelation via preloaded program, the processing unit 116 can be configured or reconfigured for use in a system 100 having any number of channels, such as one (1) channel, two (2) channels, eight (8) channels, ten (10) channels, etc. In embodiments, the sensing board has a plurality of input pins (e.g., 20), each with an individual ground and thereby, each pin can sample information from a specific detector Accordingly, the system 100 is capable of handling multiple source-detector configurations (e.g., configurations having any combination of one or more sources 104 and one or more detectors 110), as are often required in DCS applications, particularly for DCS in brain-computer interface applications. Furthermore, the processing unit 116 can perform rapid (e.g., substantially real-time) processing of signals (e.g., digitized signals). In embodiments, processing is achieved by way of GPU-assisted computational processing of the input signals using e.g., Fast-Fourier transforms to improve speed of calculations/autocorrelations.

Since the detector 110, the sampler 114, and/or the processing unit 116 are separable, for example, into distinct components, devices, systems, etc., autocorrelation of signals can be performed by a processor that is separate from the detector 110. Since the detector 110 is typically coupled to the volume 102, the system 100 enables the sampler 114 and/or the processing unit 116 to be positioned separate and/or away from the volume 102. Accordingly, portions of the system 100, such as the detector 110, can be integrated into a wearable device without requiring other portions of the system, such as the sampler 114 and/or the processing unit 116, to also be integrated into the wearable device. Accordingly, the wearable device need not bear the additional weight and power consumption associated with the sampler 114 and/or the processing unit 116. Furthermore, since the detector 110 of the wearable device and the processing unit 116 may be wirelessly connected, the detector 110, and thereby the wearable device, can be untethered from the processing unit 116. Accordingly, a person wearing such a wearable device has greater freedom of movement as compared to wearing another wearable device employing a conventional autocorrelation system that requires a hardwired connection between a detector and an autocorrelator.

For example, the system 100 can be used in conjunction with optical sensing methods to dynamically monitor and detect tissue function, structure, physiology, or blood flow therethrough. In a further example, the system 100 can be used to non-invasively and quantitatively assess changes in blood flow occurring in different regions of the cortical surface, which can provide valuable information about brain activity since the energetics of the brain is dictated by delivery and consumption of vascular oxygen. Furthermore, the system 100 may be used in conjunction with or for the development of brain-computer interfaces (BCI). To be effective in cognitive or psychological experiments, a BCI system typically requires a tomographic architecture (i.e., one with multiple source-detector channels), which in turn, requires measurement of multichannel autocorrelations. Accordingly, the system 100 can generate one or more autocorrelated signals in embodiments of the system 100 having a plurality of channels. Moreover, the system 100 can be used in conjunction with optical sensing approaches, such as near infrared spectroscopy (NIRS), for non-invasively monitoring blood oxygenation and concentration in human tissues, since systems implementing NIRS approaches sometimes include DCS signals for improved tissue sensing.

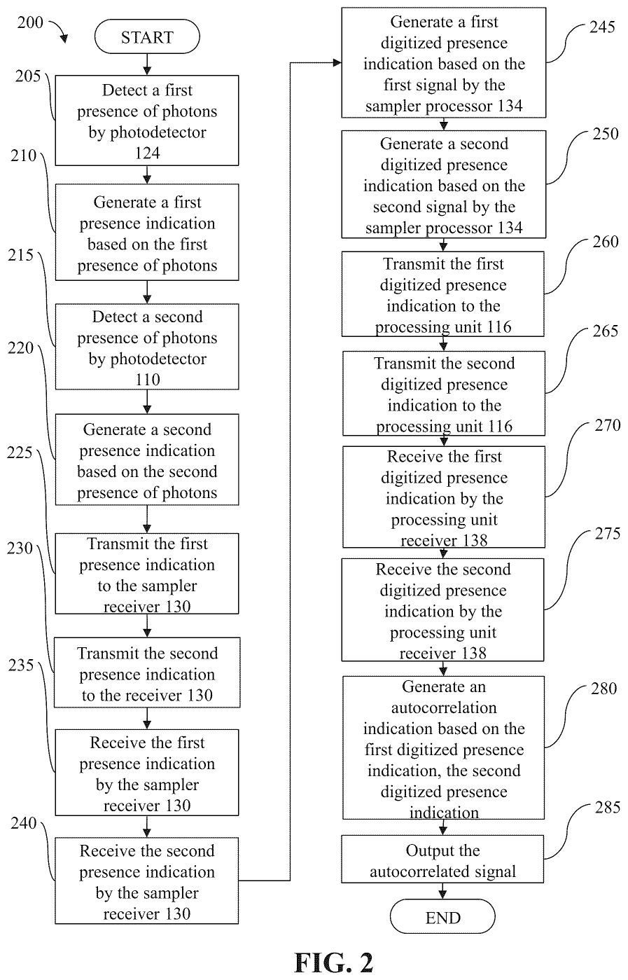

Referring now to FIG. 2, a method pertaining to autocorrelation of signals is presented. While the methodology is shown and described as being a series of acts that are performed in a sequence, it is to be understood and appreciated that the methodology is not limited by the order of the sequence. For example, some acts can occur in a different order than what is described herein. In addition, an act can occur concurrently with another act. Further, in some instances, not all acts may be required to implement a methodology described herein. An exemplary method 200 for digital autocorrelation of temporal signals is shown in FIG. 2. At step 205, the detector 110 detects a first presence or absence of photons. At step 210, the detector 110 generates a first presence indication based on the first presence or absence of photons. At step 215, the detector 110 detects a second presence or absence of photons. At step 220, the detector 110 generates a second presence indication based on the second presence or absence of photons. At step 225, the detector 110 transmits the first presence indication to the sampler 114. At step 230, the detector 110 transmits the second presence indication to the sampler 114. At step 235, the sampler 114 receives the first presence indication. At step 240, the sampler 114 receives the second presence indication. At step 245, the sampler 114 generates a first digitized presence indication based on the first presence indication. At step 250, the sampler 114 generate a second digitized presence indication based on the second presence indication. At step 260, the sampler 114 transmits the first digitized presence indication to the processing unit 116. At step 265, the sampler 114 transmits the second digitized presence indication to the processing unit 116. At step 270, the processing unit 116 receives the first digitized presence indication. At step 275, the processing unit 116 receives the second digitized presence indication. At step 280, the processing unit 116 generates an autocorrelation indication based on the first digitized presence indication and the second digitized presence indication. At step 290, the autocorrelation indication is outputted. In other embodiments, the autocorrelated signal is generated by perform FFT of the signal (1), take conjugate of FFT (2), multiply 1 with 2 and take inverse FFT (3), apply a logfilter (a smoothing function) output from 3, to get the autocorrelation. At 214, the autocorrelated signal is outputted.

In an alternative embodiment, the detector 110 is configured to generate voltages according to the TTL protocol. For example, the detector 110 can be configured to generate a voltage of around 0V to indicate no photon is detected and configured to generate a voltage of 3.3V-5V to indicate one photon is detected. In other embodiments, the detector 110 is configured to generate other signals to indicate the presence of photons at each detection. In an alternative embodiment, the detector 110 is configured to detect the presence and number of more than one photons. In such embodiment, the detector 110 is further configured to transmit the presence of more than one photons and/or the number of photons detected. In other embodiments and as discussed above, the detector 110 can be configured to be connected to multiple collection fibers or configured to have multiple photodetectors.

In an embodiment, the sampler 114 is configured to convert the voltage into binary representation of the voltage. For example, the sampler 114 is configured to convert a voltage of 0.0V (if no photon is detected) into 00 and configured to convert a voltage of 3.3V-5V (if one photon is detected) into 01 and store such information in a file with binary file format. In other embodiments, the sampler 114 can be configured to generate different signals to represent the received signals. The sampler 114 can be configured to transmit the generated digital representation of the signals as soon as it is generated or wait and transmit the generated digital representation at a predetermined aggregate. In some embodiments, the sampler 114 is configured to buffer the received signal in the sampler memory 132 before processing. Similarly, the sampler 114 can be configured to store the generated digital representation of the signals into the sampler memory 132 and/or other data storage before or after transmission. In other embodiments, the sampler 114 is configured to receive signals from more than one detectors. In such embodiments, the sampler 114 is configured to process and store the signals according to a predetermined protocol to indicate the channel of received signal.

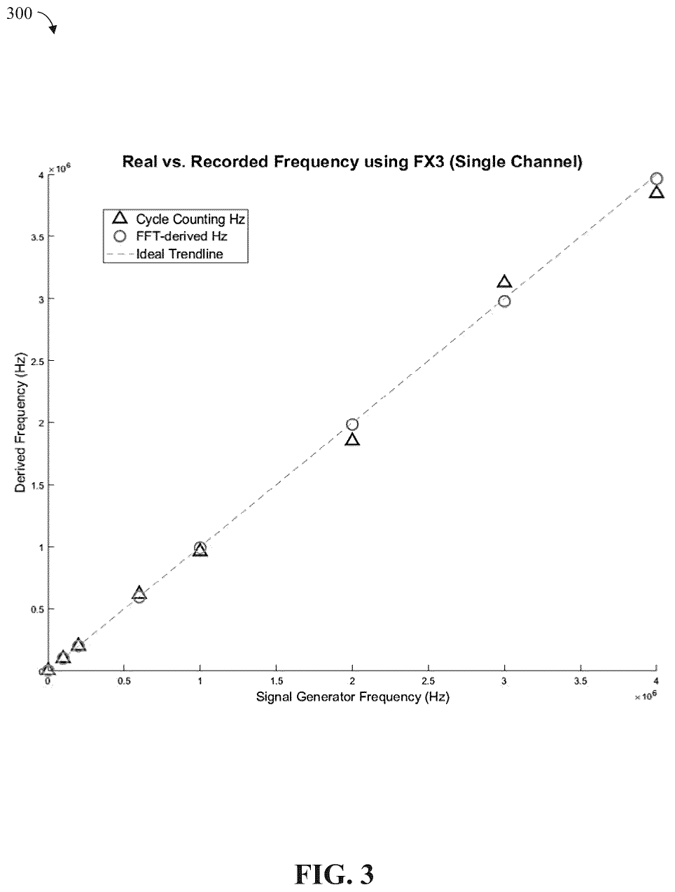

Turning now to FIGS. 3-5, exemplary test results of a system as described herein are presented in graphs 300, 400, and 500. The test results are from embodiments of a sampler 114. In certain embodiments, a sampling frequency of the sampler 114 (i.e., a derived frequency or an experimentally determined frequency) can be affected by a total length of data collection time (e.g., a sampling period, a sum total of sampling periods) and/or the number of input signal channels in the system 100. Referring to the graphs 300, 400, and 500, the x-axis of each of the graphs corresponds to a known frequency of a signal generated by a signal generator, such as a standard, laboratory-grade function generator capable of controlling input voltage, repetition rates, frequency, and waveform of the signal. The y-axis of each of the graphs corresponds to a derived frequency (i.e., an experimentally determined frequency). As noted in the legend of each of the graphs, the experimentally determined frequency can be determined by the sampler 114 performing a cycle counting methodology as described herein. In an embodiment, cycle counting may be achieved by comparing how long the signal was high relative to how long was the signal low for a periodic input signal of square waves (of known input frequency) that are e.g., sampled for a million times longer than the time-period of the input square wave. In this way the frequency of the sampled input may be computed. This allows for verification of the control signals. The fidelity (i.e., the accuracy of an experimentally determined frequency relative to a known frequency) of the cycle counting methodology is compared to a conventional FFT-derived methodology. Frequencies experimentally determined by the cycle counting methodology (marked by triangles in the graphs) and frequencies experimentally determined by the conventional FFT-derived methodology (marked by circles in the graphs) are compared to an ideal trendline, as shown by a dashed line on the graphs. The ideal trendline has a slope of one (1). In other words, the ideal trendline illustrates an undegraded fidelity such that for a known frequency of a signal generated by a signal generator, an experimentally determined frequency exactly matches the known frequency.

Now referring specifically to FIG. 3, the graph 300 shows that for an embodiment of a system 100 having a single channel (e.g., a single pair comprising a source 104 and a detector 110), the cycle counting methodology (e.g., employed by the sampler 114) yields experimentally determined frequencies that are substantially similar to the known frequencies generated by a signal generator. As shown in the graph 300, the cycle counting methodology may have an especially high fidelity when sampling at a frequency less than about 20 MHz, including 10 MHz.

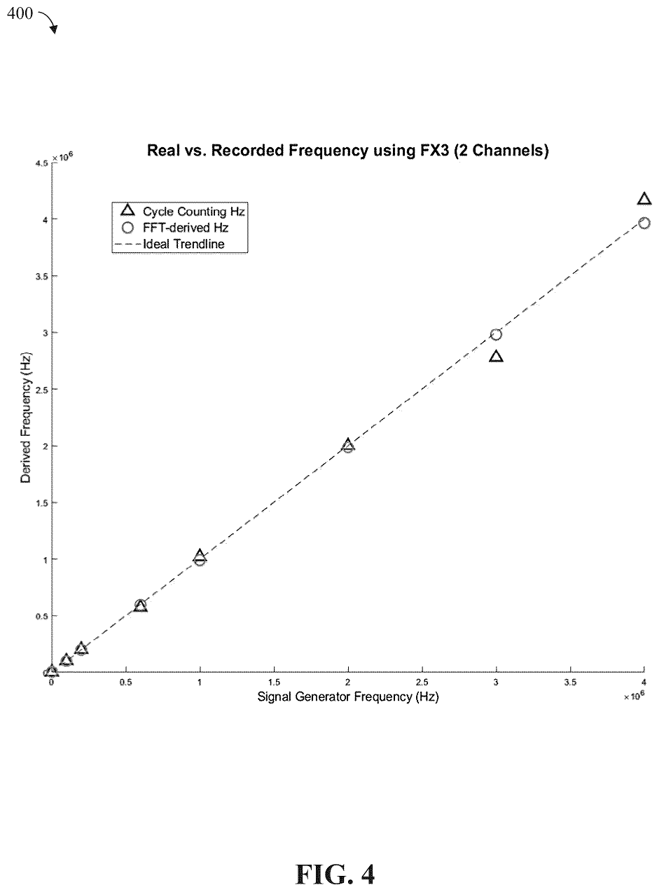

Turning to FIG. 4, the graph 400 shows that for an embodiment of a system 100 having two channels (e.g., two pairs where each pair comprises a source 104 and a detector 110), the cycle counting methodology (e.g., employed by the sampler 114) yields experimentally determined frequencies that are substantially similar to the known frequencies generated by a signal generator. As shown in the graph 400, the cycle counting methodology may have an especially high fidelity when sampling at a frequency less than about 30 MHz and results are not impacted if sensing from one or more channels. The fidelity of collected data is not impacted.

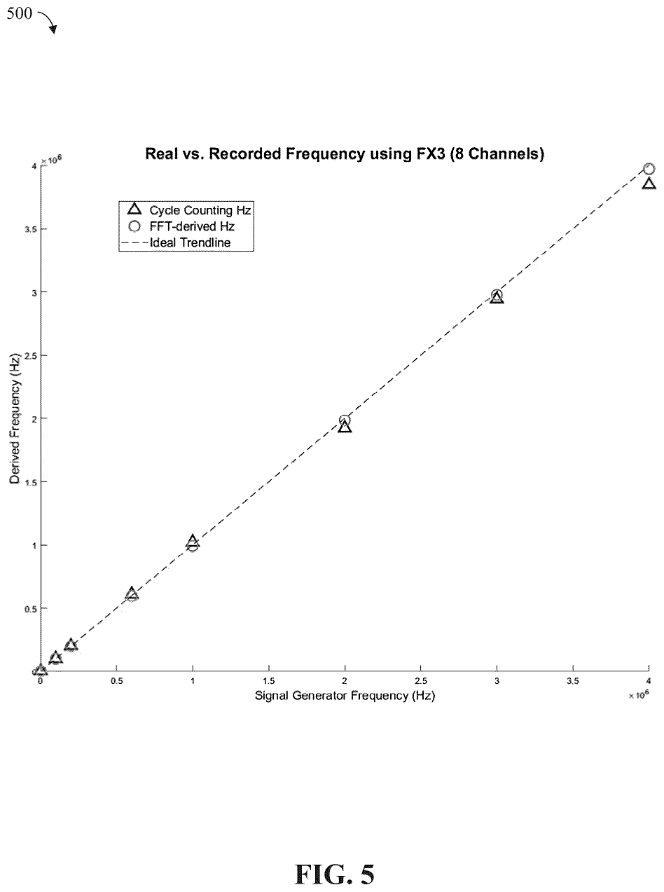

With reference to FIG. 5, the graph 500 shows that for an embodiment of a system 100 having eight channels (e.g., eight pairs where each pair comprises a source 104 and a detector 110), the cycle counting methodology (e.g., employed by the sampler 114) yields experimentally determined frequencies that are substantially similar to the known frequencies generated by a signal generator. As shown in the graph 500, the cycle counting methodology may have an especially high fidelity when sampling at a frequency less than about 40 MHz. Accordingly, embodiments of the system 100, such as embodiments that employ the cycle counting methodology and/or the sampler 114, are configured to sample a signal at a frequency of at least about 10 MHz, at least about 20 MHz, at least about 30 MHz, at least about 40 MHz, etc.

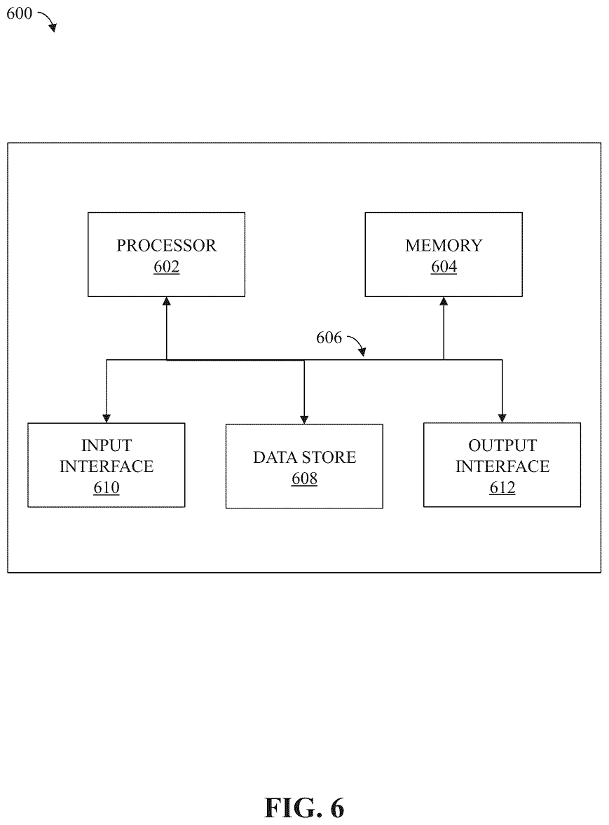

Referring now to FIG. 6, a high-level illustration of an exemplary computing device 600 that can be used in accordance with the systems and methodologies disclosed herein is illustrated. For instance, the computing device 600 may be used in a system for autocorrelating temporal signals (e.g., system 100). The computing device 600 includes at least one processor 602 that executes instructions that are stored in a memory 604. The instructions may be, for instance, instructions for implementing functionality described as being carried out by one or more components discussed above or instructions for implementing one or more of the methods described above. The processor 602 may access the memory 604 by way of a system bus 606.

In alternative embodiments, the computing device 600 is configured to receive and/or process the digital representation of the signals sent by the sampler 114. In alternative embodiments, the computing device 600 is configured to load the configurations into the detector 110 and/or the sampler 114. In some embodiments, the computing device 600 is configured to change the settings in the sampler 114 (including but not limited to sampling period and/or lag time) and/or the detector 110. In some embodiments, the computing device 600 is configured to convert and/or store the digital representation of the signals sent by the sampler 114.

The computing device 600 additionally includes a data store 608 that is accessible by the processor 602 by way of the system bus 606. The data store 608 may include executable instructions.

The computing device 600 also includes an input interface 610 that allows external devices to communicate with the computing device 600. For instance, the input interface 610 may be used to receive instructions from an external computer device, from a user, etc. The computing device 600 also includes an output interface 612 that interfaces the computing device 600 with one or more external devices. For example, the computing device 600 may display text, images, etc. by way of the output interface 612.

It is contemplated that the external devices that communicate with the computing device 600 via the input interface 610 and the output interface 612 can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 600 in a manner free from constraints imposed by input devices such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 600 may be a distributed system. Thus, for instance, several devices may be in communication by way of a network connection and may collectively perform tasks described as being performed by the computing device 600.

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. Disk and disc, as used herein, include compact disc (CD), laser disc, optical disc, digital versatile disc (DVD), floppy disk, and Blu-ray disc (BD), where disks usually reproduce data magnetically and discs usually reproduce data optically with lasers. Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionally described herein can be performed, at least in part, by one or more hardware logic components. For example, and without limitation, illustrative types of hardware logic components that can be used include Application-specific Integrated Circuits (ASICs), Application-specific Standard Products (ASSPs), System-on-a-chip systems (SOCs), Complex Programmable Logic Devices (CPLDs), etc.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

Reference is made to one or more articles, patents, patent applications, or other publications, the entire content of which are expressly incorporated herein as if recited in their entirety.

1. A system for generating autocorrelation indications, the system comprising:
a detector configured to:
detect a first presence or absence of a first photon,
generate a first presence indication based on the first presence or absence of the first photon,
transmit the first presence indication,
detect a second presence or absence of a second photon,
generate a second presence indication based on the second presence or absence of the second photon, and
transmit the second presence indication;
a sampler configured to:
receive the first presence indication from the detector,
receive the second presence indication from the detector,
generate a first digitized presence indication based on the first presence indication, and
generate a second digitized presence indication based on the second presence indication; and
a processing unit comprising a processor, a receiver, and a non-transitory computer-readable medium storing machine executable instructions, wherein when executed by the processor, cause the processor to perform acts comprising:
receiving the first digitized presence indication and the second digitized presence indication, and
generating an autocorrelation indication based on the first digitized presence indication and the second digitized presence indication.

2. The system of claim 1, wherein the detector comprises a photodetector configured to generate the first and second presence indication based on if a photon is detected by the photodetector.

3. The system of claim 2, wherein the detector is a single photon avalanche diode (SPAD).

4. The system of claim 1, wherein the processing unit is configured to generate the autocorrelated signal based on a plurality of pairs of first and second digitized signals.

5. The system of claim 4, wherein the detector comprises a plurality of photodetectors, wherein each pair of first and second digitized presence indications corresponds to a photodetector of the plurality of photodetectors.

6. The system of claim 1, wherein the detector is configured to generate a voltage of around 0.0V if no photon is detected and a voltage of 3.3V to 5.0V if a photon is detected.

7. The system of claim 1, wherein the sampler is configured to sample the presence indication at a frequency of at least about 10 MHz.

8. The system of claim 1, wherein the sampler is configured to sample the presence indication for a predetermine period of time.

9. The system of claim 1, wherein the sampler is configured to generate a binary indication based on if a photon is detected by the detector.

10. The system of claim 1, wherein the sampler and the processing unit are in electrical communication by way of a USB connection, and the sampler is configured to transmit the digitized presence indication to the processing unit by way of the USB connection.

11. The system of claim 1, wherein the sampler and the processing unit are in wireless communication.

12. The system of claim 11, wherein the detector and the sampler are in wireless communication.

13. The system of claim 11, wherein the processing unit is a cloud-based processing unit.

14. The system of claim 1, wherein the detector comprises more than one photodetector and is configured to transmit more than one presence indications, the sampler is configured to generate more than one digitized presence indications, and the processing unit is configured to generate autocorrelation indications based on more than one multiple digitized presence indications.

15. The system of claim 1, wherein the detector is configured to detect the presence or amount of other substance.

16. A method for generating autocorrelation indications, the method comprising:
detecting a first presence or absence of a first photon by a detector;
generating a first presence indication based on the first presence or absence of the first photon by the detector;
transmitting the first presence indication by the detector;
detecting a second presence or absence of a second photon by the detector;
generating a second presence indication based on the first presence or absence of the second photon by the detector;
transmitting the second presence indication by the detector;
receiving the first presence indication by a sampler;
receiving the second presence indication by the sampler;
generating a first digitized presence indication based on the first presence indication by the sampler;
generating a second digitized presence indication based on the second presence indication by the sampler;
receiving the first digitized presence indication and the second digitized presence indication by a processing unit;
generating an autocorrelation indication based on the first digitized presence indication and the second digitized presence indication; and
outputting the autocorrelation indication.

17. The method of claim 16, wherein the method, instead of detecting the presence of photons, detects the presence or amount of other substance.

18. The method of claim 16 further comprises compressing the digitized presence indication by the sampler and decompressing the digitized presence indication by the processing unit.
